(19) United States
(12) Patent Application Publication
(10) Pub. No.: US 2016/0256589 A1
(43) Pub. Date: Sep. 8, 2016

(54) SYSTEM AND METHOD FOR DISPENSING LIQUIDS

(71) Applicants: Tsafrir SASSON, Kibbutz Ma'agan Michael (IL); SCENTCOM LTD., Lehavot Haviva (IL)

(72) Inventor: Tsafrir SASSON, Kibbutz Ma'agan Michael (IL)

(21) Appl. No.: 15/026,640

(22) PCT Filed: Sep. 22, 2014
(86) PCT No.: PCT/IL2014/050843

§ 371 Date: Apr. 1, 2016

Related U.S. Application Data
(60) Provisional application No. 61/885,127, filed on Oct. 1, 2013.

(51) Int. Cl. A61L 9/14; A61L 9/013; B05B 17/06

(52) U.S. Cl. CPC A61L 9/14; B05B 17/0684; A61L 9/013; A61L 2209/132

(57) ABSTRACT

A system for dispensing liquids comprising: a reservoir containing a fluid, a wick which is in contact with the fluid, and, an ultrasonic atomizer, wherein a portion of the wick in contact with the ultrasonic atomizer. The wick is made from an inorganic material which is preferably metal, and is in contact with the ultrasonic atomizer in a spring-like manner.

### FIELD OF THE INVENTION

The present invention relates to the field of systems for dispensing liquids, and more particularly to a wicking formation which supports the supply of liquid to a vibratory mechanism for dispensing liquids into the ambient.

### BACKGROUND OF THE INVENTION

Systems for dispensing liquids into the ambient are known. US Patent Application No. 2011/0266359A1 discloses a device for controllably dispersing liquids. The mechanism that propels the scent bearing liquid into the ambient employs an energized plate stubbed with an array of micro plugs that thrust forcibly into holes of a parallel plate such that the micro plugs can drive out the scent bearing liquid through the array of holes.

The mechanism that propels the liquid drains the liquid provided by a wick, while the wick draws the liquid from a receptacle. Therefore, the wick serves as a conduit of liquid from the receptacle to a dispersing module.

In order to better clarify the elements of the system according to the present invention, a reference to an illustration of a prior art system is first made in FIG. 1. Thus, FIG. 1 describes schematically the translocation of liquid from a reservoir 32, which in a specific case is a scent bearing liquid. The liquid moves from the reservoir by capillary forces implemented by a wick 34 to a forwarding module 36 which further translocates the liquid to a dispersing module 38.

One end of the wick 34, typically forming a lower end, is soaked in a reservoir of liquid such as a scent bearing oily liquid, while its upper end is set against a forwarding module 36, such that a continuum of liquid is kept from the container to the forwarding module 36. The capillary structure of the wick 34 provides the surface tension for drawing the liquid, typically against gravity, and bringing it to the forwarding module 36.

There are commercial electronic systems for dispersing scented liquids by heating the liquids, or, by using diluted scents that are diluted with water or with VOC (Volatile Organic Compounds). In these systems, the dispersing action functions much easier than with systems that use directly the oily odorous concentrate. However, a disadvantage of water based systems is that they require about a 10 times larger receptacle for storing the diluted odorous concentrate to be used, comparing to receptacles storing the odorous concentrate.

In these cases, when a larger receptacle has to be used, it forces other changes in the system, for example, stiffness, size, possible locations, possible uses, which forces a multitude of restrictions upon the system.

If it is not desired or practically not possible to enlarge the storing receptacle, then, another disadvantage is that the usable time of such systems will be 10 times shortened, a fact that also prevents satisfactory, useful and practical use of the system.

Other disadvantages of current commercial systems are that heating the essential oils changes the aroma and forms VOC, and, using essential oils with VOC.

A disadvantage of known organic wicks for translocating oily odorous concentrates to ultrasonic atomizers is that they can react with essential oil and tend to disintegrate, and, loose the mechanical properties and stability. In which case, they could not provide a satisfactory functioning of the system as a steady conductor of oil to the atomizer

Another disadvantage of known organic wicks is that they are affected by temperature variations. The temperature variations cause expansion and contraction, variations in dimensional stability and in the stiffness of the wicks.

The existing organic materials that are used as wicks for ultrasonic atomizers are porous materials like, e.g., sintered polymeric materials (available from POREX™), polymeric fibers (nylon, PET), organic fibers (polymeric fibers), inorganic fibers (silicone), natural fibers (cotton fibers), cellular plastic (sponge or foam, like polyurethane foam). These materials may be used as conductors of the oily extract, or, as accessory parts that press on the membrane of the ultrasonic atomizer However, these organic materials cannot be used simultaneously to conduct the oily extract and to press on the membrane as well.

It is the object of the present invention to provide a system for containing, translocating and dispersing odorous liquids that significantly reduces or overcomes the aforementioned disadvantages.

It is a further object of the present invention to provide a system for containing, translocating and dispersing odorous liquids that is electronically applied and controlled for intensity and duration.

It is still a further object of the present invention to provide a system for containing, translocating and dispersing odorous liquids that prevents the phenomenon of fluid flooding in the space beneath the atomizer

It is yet a further object of the present invention to provide an inorganic wick that is not affected by oily extracts.

It is still yet an object of the present invention to provide an inorganic wick that can be used with corrosive materials.

It is still another object of the present invention to provide an inorganic wick that can be used with high viscosity materials.

It is still yet another object of the present invention to provide a metallic wick for ultrasonic atomizers.

It is another object of the present invention to provide an inorganic wick for ultrasonic atomizers having a retractable feeding providing constant pressure against a membrane of the ultrasonic atomizer

It is a further object of the present invention to provide a metallic wick for ultrasonic atomizers having a spring-like formation that reduces loads on the membrane of the atomizer.

It is also another object of the present invention to provide a system for containing, translocating and dispersing odorous liquids, which can use the natural extract of the odors without any additions of organic conductors such as alcohol and acetone, a fact that provides many regulatory advantages as well as safety and health benefits.

### SUMMARY OF THE INVENTION

In accordance with the present invention there is provided a system for dispensing liquids comprising:

a reservoir containing a fluid,

a wick, at least a portion of the wick in contact with the fluid,

an ultrasonic atomizer, at least a portion of the wick in contact with the ultrasonic atomizer, wherein;

the wick is made from an inorganic material.

Typically, the wick is made of metal.

If desired, the wick has a form of a mesh connected to a spring.

Preferably, the spring is made of metal.

Advantageously, the wick having a spring conductivity with the ultrasonic atomizer

Typically, the wick has a bend in an upper portion thereof.

Advantageously, the wick functions as a fluid conductive agent, and, as a spring-like element.

Practically, the fluid is an oily scent bearing liquid.

Preferably, the wick is made of an inorganic material.

Typically, the wick is made of metal.

Advantageously, the wick provides a spring conductivity.

If desired, the wick is made of mesh connected to a spring.

In most cases, the spring is made of metal.

Typically, the wick is formed with a bend in an upper portion thereof.

Advantageously, the wick functions as a fluid conductive agent, and, as a spring-like element.

If desired, the wick is made of a metal mesh.

Further if desired, the wick is made of a woven mesh.

Still further if desired, the wick is made of a sintered wire.

Still if desired, the wick is made of a metal braid.

Still further if desired, the wick is made of a sintered mesh.

Still yet if desired, the wick is made of a microfluidic element.

In accordance with some embodiments, the wick is made of a plastic.

Still in accordance with some embodiments, the wick is made of ceramic.

Still further in accordance with some embodiments, the wick is made of silicone.

### BRIEF DESCRIPTION OF THE DRAWINGS

For a better understanding of the present invention and to show how the same may be carried out in practice, reference will now be made to the accompanying drawings, in which:

FIG. 1 is a schematical block diagram description of a prior art system for translocating a liquid from a reservoir;

FIG. 2 is a schematical block diagram system of manufacturing steps of a wick according to the present invention;

FIG. 3 is a perspective view of a wick according to the present invention within a framework of a dispenser; and

FIG. 4 is a schematical representation of the concept of the starvation feed of the system according to the present invention.

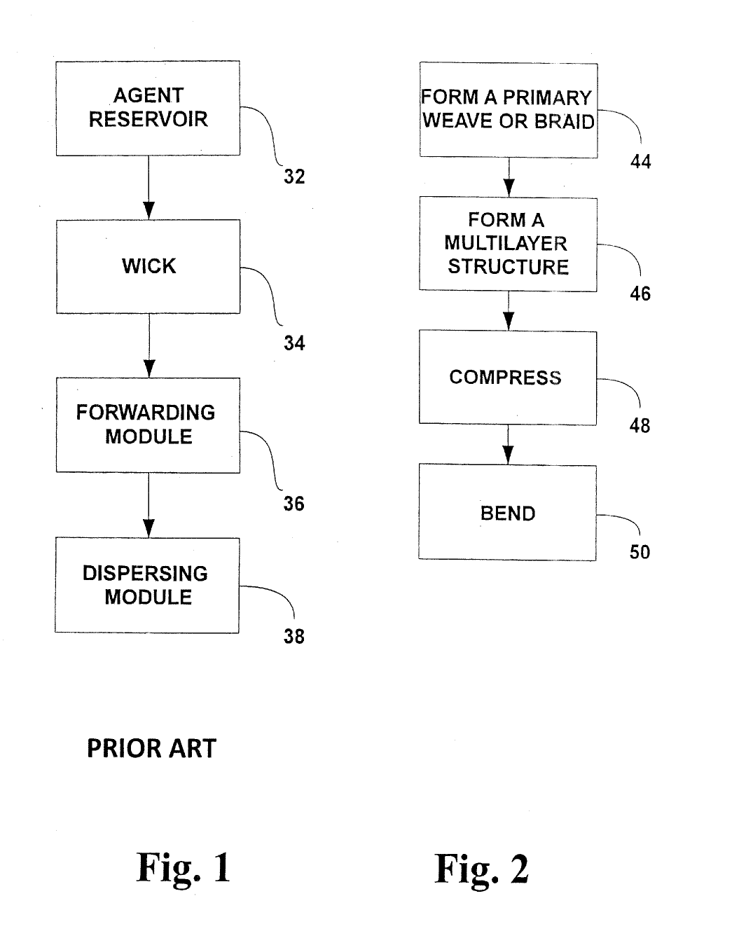

### DESCRIPTION OF PREFERRED EMBODIMENTS

In accordance with the present invention, odorous liquids are stored in containers from which they are drawn out by wicks due to capillary forces acting thereon. Such odorous liquids may be pre-formulated in the containers or may be formulated whilst dispersed.

The present invention provides novel formations of wicks for use in a variety of usage fields. Thus, the novel formations of wicks may be used for inhalation in medical fields, for atmosphere humidifying in daily care, and, for scent dispensing applications.

In accordance with the present invention a wick is made either as a mesh structure or as a braided structure. Both such structures provide the interstices of the physical infrastructure. The mesh structure is easier explained as it is made by weaving of fibers in the shape of a sheet, typically forming a right angle between the lateral and the longitudinal threads or fibers. These primary formations, are then used for superstructures in one or several stages. For example, a formed sheet can be curled to form a tube, or a different kind of three dimensional structure. Often a multilayered tube is formed for increasing the number of interstices. Another type of mesh wick is a perforated plate type. At this type, a thin perforated plate of solid material is bent to form a tube or another structure. In these forms, multilayered formations provide more interstices as capillary gaps are formed between layers.

The braided primary structure is typically more complicated than the structure of a mesh primary structure since the weavings cannot be carried out in a sheet form, which is then folded to make a superstructure. In accordance with the present invention, braided wicks are formed into a superstructure, multilayered and such. One prominent feature of the braided wick is its tendency for relatively low stiffness in the direction of its main axis. This feature will be later discussed in more detail in connection with the materials from which the wick is made.

Thus, the formation of a wick according to the teachings of the present invention can be formed according one of the following modules:

(1) A mesh structure is formed on a metallic leaf-like spring having an arcuate shape. The mesh structure is connected or attached to the spring.

(2) A long braded tube, formed as a round sleeve having a spring-action form.

(3) A sintered wire/woven mesh. This is a multilayer mesh structure that enables to produce an array of layers, with an external support in order to produce a defined structure.

(4) A thin plate, with holes or channels that enable capillarity. This may be a perforated plate or sheet.

(5) A wick element that is based on capillary fluid flow through narrow channels, either open or closed channels. It is possible to produce a metal spring having micro tunnels. Such a wick element may be produced from metal, plastic, ceramic, or silicone.

It should be noted that directional terms appearing throughout the specification and claims, e.g. “forward”, “rear”, “upper”, “lower” etc., are used as terms of convenience to distinguish the location of various surfaces relative to each other. These terms are defined with reference to the figures, however, they are used for illustrative purposes only, and are not intended to limit the scope of the appended claims.

Reference is now made to FIG. 2 which shows an exemplary sequence of manufacturing steps of a wick. At a first step 44 the primary woven sheet or a perforated solid sheet are made. At a second step 46 a multilayered structure is formed. At a third step 48, the multilayered structure is compressed. At a fourth step 50 a bend is introduced, for example, to permit extended contact between the wick and the nebulizer/atomizer

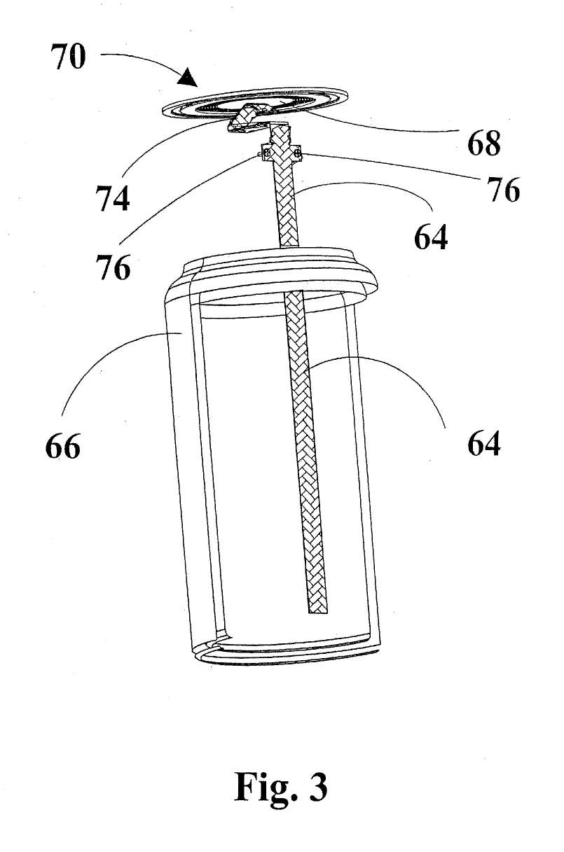

FIG. 3 shows a wick 64 made in accordance with the present invention and shown within the framework of a dispenser, simplified for the sake of demonstrability. The wick 64 runs substantially from the bottom of a liquid receptacle 66 through a cap assembly (not shown) until it reaches its peak and barely touches a bottom 68 of an atomizer 70. In order to increase the interface area with the atomizer 70, a bend 74 is formed at the top of the wick 64. In addition, two wings 76 are formed in the wick 64 to facilitate fixing the wick 64 to the cap (not shown).

The bend 74 that is formed at the top of the wick 64 enables to reduce loads on the membrane at the bottom 68 of the atomizer 70. Furthermore, the bend 74 provides a relatively stable pressure, though very small, on the membrane. By this construction, the relatively stable pressure is obtained without being dependent on the relative position between the wick 64 and the membrane. The relative position may vary according to variations of dimensions of a new receptacle 66 when replaced with an old receptacle, or, due to misplacing or differently placing the new receptacle 66 by an operator.

The bend 74 may be formed as shown, or, have other shapes. For example, the bend may be formed as a large arcuate shape having a controllable pre-determined stiffness and flexibility, and, enabling its upper portion to controllably barely touch the bottom 68 of the atomizer 70.

A most prominent feature of a wick in accordance with the present invention are the materials from which the wick is made. The materials can be divided into two main groups: (1) metals, and (2) ceramics or inorganic polymer structures. However, the intercalation of organic fibers in a wick of the invention is not precluded.

A list of applicable metals may include, but not limited to, cobalt-chromium alloy, nitinol, stainless steel, specifically 316 with or without passivation. It has been observed that using metal fiber wicks is favorable with respect to prevention of the phenomenon of fluid flooding in the space beneath the atomizer, when the lower part of which was referred to above as a forwarding module.

The best way to describe this effective mode of action which the metallic wick facilitates is “starvation feed”. This means that although the continuum from the container to the atomizer, or forwarding module, is efficient, no stacking of liquid is observed, no flooding of any part of the continuum occurs, and no overflow takes place. The liquid supply rate can be controlled in the design stage by changing structural parameters according to need. For example, an increase in the number of layers would increase the buffer capacity of the wick etc. The preferred mode of operation would be a “starvation feed” mode.

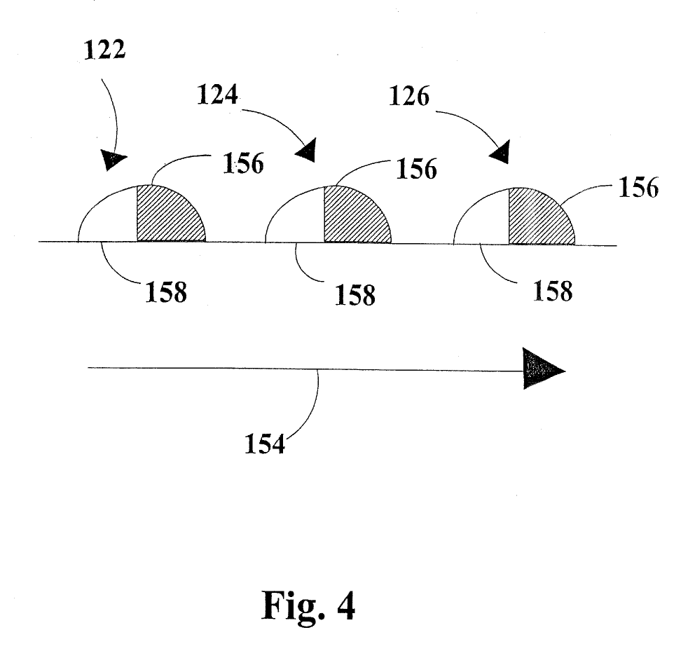

Reference is made now to FIG. 4 to explain the concept of the “starvation feed”. The dispensation of liquid using the technology as explained in US Patent Application No. 2011/0266359A1 usually takes place in pulses. A first pulse 122, a second pulse 124, and a third pulse 126 are an exemplary sequence of pulses, taking place on a time arrow 154. Each pulse has a “wet” sector 156 and a “dry” sector 158 with a transition area therebetween that is not shown.

Functionally, as the dispensing pulse starts, the liquid is outwardly dispensed, but since the translocation in the wick 64 is limited in rate, it dries up before the pulse ends, forming the dry sector, in which the wick, the atomizer and the ambient volume of air around the atomizer dry up, preparing for the next pulse.

The use of metal, inorganic, or imperforated wicks, enables to better design the starvation feed, whose functioning described above. In these wicks, the oily fluid may flow only on the surface thereon and cannot get into the material of which the wick is made. In this way, flooding is prevented, and the dispensing can be designed such that each operation utilizes the entire fluid that is available on the surface of the wick that is in contact with the bottom 68 of the atomizer 70. It should be clear that by using the phrase “on the surface of the wick”, it includes also the envelope of the entire formation of the wick, including the spaces between the mesh, the braid, and the like. However, is excludes the internal volume of the wires of the mesh since they are not absorbable and cannot be impregnated by the fluid.

The metal wicks are less prone to environmental influences, referring mainly to ambient temperatures and humidity. The use of metal fibers for manufacturing wicks provides structures having physical properties resembling those of a spring. In this respect, braided wicks made of metal fibers offer less rigidity and lower spring constant along the axis of a wick. This facilitates the use of metallic braids to be set against parts of the dispensing modules which may be specifically mechanically vulnerable. Generally, metallic wicks are resilient and therefore are more tolerant to variability in geometrical features, specifically, the distance to the atomizers.

Although the present invention has been described to a certain degree of particularity, it should be understood that various alterations and modifications could be made without departing from the spirit or scope of the invention as hereinafter claimed.

For example, the bend of the wick does not have to be formed from relatively straight segments and the entire bend may have a circular form.

1. A system for dispensing liquids comprising:
a reservoir containing a fluid,
a wick, at least a portion of the wick in contact with the fluid,
an ultrasonic atomizer, at least a portion of the wick in contact with the ultrasonic atomizer, wherein;
the wick is made from an inorganic material.

2. The system for dispensing liquids according to claim 1, wherein:
the wick is made of metal.

3. The system for dispensing liquids according to claim 2, wherein:
the wick has a form of a mesh connected to a spring.

4. The system for dispensing liquids according to claim 3, wherein:
the spring is made of metal.

5. The system for dispensing liquids according to claim 1, wherein:
the wick having a spring conductivity with the ultrasonic atomizer.

6. The system for dispensing liquids according to claim 1, wherein:
the wick has a bend in an upper portion thereof.

7. The system for dispensing liquids according to claim 1, wherein:
the wick functions as a fluid conductive agent, and, as a spring-like element.

8. The system for dispensing liquids according to claim 7, wherein:
the fluid is an oily scent bearing liquid.

9. A wick for an ultrasonic atomizer, wherein:
the wick is made of an inorganic material.

10. The wick for an ultrasonic atomizer according to claim 9, wherein:
the wick is made of metal.

11. The wick for an ultrasonic atomizer according to claim 9, wherein:
the wick provides a spring conductivity.

12. The wick for an ultrasonic atomizer according to claim 9, wherein:
the wick is made of mesh connected to a spring.

13. The wick for an ultrasonic atomizer according to claim 12, wherein:
the spring is made of metal.

14. The wick for an ultrasonic atomizer according to claim 9, wherein:
the wick is formed with a bend in an upper portion thereof.

15. The wick for an ultrasonic atomizer according to claim 9, wherein:
the wick functions as a fluid conductive agent, and, as a spring-like element.

16. The wick for an ultrasonic atomizer according to claim 9, wherein:
the wick is made of a metal mesh.

17. The wick for an ultrasonic atomizer according to claim 9, wherein:
the wick is made of a woven mesh.

18. The wick for an ultrasonic atomizer according to claim 9, wherein:
the wick is made of a sintered wire.

19. The wick for an ultrasonic atomizer according to claim 9, wherein:
the wick is made of a metal braid.

20. The wick for an ultrasonic atomizer according to claim 9, wherein:
the wick is made of a sintered mesh.

21. The wick for an ultrasonic atomizer according to claim 9, wherein:
the wick is made of a microfluidic element.

22. The wick for an ultrasonic atomizer according to claim 9, wherein:
the wick is made of a plastic.

23. The wick for an ultrasonic atomizer according to claim 9, wherein:
the wick is made of ceramic.

24. The wick for an ultrasonic atomizer according to claim 9, wherein:
the wick is made of silicone.
